(19) United States
(12) Patent Application Publication
(10) Pub. No.: US 2026/0224379 A1
(43) Pub. Date: Aug. 6, 2026

(54) COVERED STENT

(71) Applicant: BCM Co., LTD., Gyeonggi-do (KR)

(72) Inventor: Byung Cheol MYUNG, Gyeonggi-do (KR)

(21) Appl. No.: 19/083,450

(22) Filed: Mar. 19, 2025

(30) Foreign Application Priority Data
Feb. 4, 2025 (KR) 10-2025-0013464

(51) Int. Cl. A61F 2/90; A61L 31/02; A61L 31/10

(52) U.S. Cl. CPC A61F 2/90; A61L 31/022; A61L 31/10; A61F 2210/0014; A61F 2210/0076; A61L 2400/16; A61L 2420/08

(57) ABSTRACT

Disclosure relates to a covered stent deformed to match a shape of a curved lumen to expand a lesion to maintain a passage in the lumen. For the covered stent including a body stent and space parts, and expansion tube portions at opposite ends of the body stent, a first polytetrafluoroethylene (PTFE) film covers an outer surface of the body stent, a second PTFE film covers outer surfaces of the expansion tube parts and outer surfaces of opposite end parts of the first film, a third PTFE film covers inner surfaces of the expansion tube parts and inner surfaces of opposite end parts of the body stent, and extend in a longitudinal direction of the body stent longer than the second film, and the first to third films are thermally bonded, and a gap space is formed between a non-bonded portion of the first film, and the body stent.

### CROSS REFERENCE TO RELATED APPLICATION

The present application claims priority to Korean Patent Application No. 10-2025-0013464, filed Feb. 4, 2025, the entire contents of which are incorporated herein for all purposes by this reference.

### BACKGROUND OF THE INVENTION

### Field of the Invention

The present disclosure relates to a covered stent expanding a stenotic or occlusive lesion in a lumen of the human body to maintain a passage in the lumen and, more particularly, to a covered stent capable of being easily positioned within a curved lumen between the esophagus and the stomach, a curved lumen between the stomach and the duodenum, and a curved lumen of the large intestine.

### Description of the Related Art

In general, when a stenotic or occlusive lesion occurs in a lumen of the body, such as the biliary tract, esophagus, ureters, or large intestine of the human body, the inherent function of said lumen to move fluids, bile, urine, and food is decreased.

Therefore, the lumen narrowed by the stenotic or occlusive lesion was expanded by inserting a stent into the lumen.

As shown in FIGS. 1 and 2, a first stent having expansion tube parts at opposite portions is an uncovered stent in which a plurality of intersection parts is formed from wires crossing each other and a plurality of space parts formed among the wires is exposed. The first stent tends to maintain a linear shape as having a strong recovery force due to the intersection parts, so it is difficult to allow the stent to remain covered.

In this regard, a stent of the patent document is formed by wire crossing or laser cutting and has a strong recovery force like the first stent, so it has difficulty in maintaining a curved state.

For this reason, as shown in FIGS. 3 to 5, when the first stent is positioned within a curved lumen between the esophagus and the stomach, a curved lumen between the stomach and the duodenum, a curved lumen of the large intestine, where lesion portions occur each, the first stent has difficulty in maintaining a curved state due to a strong recovery force that recovers the stent to a linear state.

Furthermore, since the curved lumen is brought into strong contact with the first stent that is to be recovered to the linear state, there is concern that inflammation occurs, and since the lesion portion intrudes into the space parts, a passage in the lumen is narrowed.

Furthermore, when the stent in the patent document is positioned within the curved lumen, the stent also has a strong recovery force that recovers the stent to a linear state, so the stent has difficulty in maintaining a curved state.

As shown in FIGS. 6 and 7, to compensate for the problem, a second stent with expansion tube parts at opposite side parts includes a plurality of hook parts and a plurality of cross parts, and the plurality of hook parts and the plurality of cross parts are formed by weaving wires to be caught in a hook shape or crossing the wires.

For this reason, a recovery force of the second stent is weakened due to the hook parts, and the second stent may be maintained in a curved state when the second stent is positioned within the curved lumen.

However, the second stent is an uncovered stent in which a plurality of space parts formed among the wires is exposed, and as shown in FIG. 8, as the lesion portion introduces into the space parts, the passage of the lumen is narrowed.

For this reason, the second stent can only be used with limitation to the curved lumen.

As shown in FIGS. 9 and 10, unlike the first and second stents that are uncovered stents, a third stent that is a covered stent is provided to prevent the lesion from intruding into the stent.

The third stent with expansion tube parts at the opposite side parts is covered with a polytetrafluoroethylene (PTFE) film formed by PTFE thermal bonding, and covers space parts formed among wires.

However, a plurality of cross parts and a plurality of the hook parts formed in the third stent are fixed to the PTFE film and are not moved, i.e., have no displacement. The third stent tends to maintain a linear state, as having a strong recovery force due to the PTFE film, so the third stent has difficulty in maintaining a curved state.

As shown in FIGS. 11 and 12, when the third stent is positioned within the curved lumen, it is possible to prevent the lesion from intruding into the third stent, but there is difficulty for the third stent to maintain a curved state due to a strong recovery force recovering the third stent to a linear state, and there is a concern that inflammation may occur because the curved lumen is brought into strong contact with the third stent that is to be recovered to the linear state.

Furthermore, the third stent covered with the PTFE film is bent at a curved portion of the lumen, and a curved portion of the third stent is subjected to stress for a long time to cause broken wires, and there is a concern that the third stent is broken.

As shown in FIGS. 13 and 14, instead of the PTFE film, the third stent covered with a silicone film by silicone dipping is positioned within the curved lumen.

However, likewise, the third stent covered with the silicone film also has difficulty in maintaining a curved state due to a strong recovery force recovering the third stent to a linear state, and there is a concern that inflammation may occur because the curved lumen is brought into strong contact with the third stent that is to be recovered to the linear state.

Furthermore, the third stent covered with the silicone film is bent at a curved portion of the lumen, and a curved portion of the third stent is subjected to stress for a long time to cause broken wires, and there is a concern that the third stent is broken.

For this reason, the third stent that is a covered stent can only be of limited use to the curved lumen.

There is a need for a covered stent that is improved from the problems of the related art.

### DOCUMENT OF RELATED ART

### Patent Document

Patent Document 1: Korean Patent No. 10-1493485 (Date of Registration: Feb. 9, 2015)

### SUMMARY OF THE INVENTION

Accordingly, an objective of the present disclosure is to provide a covered stent that expands a stenotic or occlusive lesion occurring in a lumen in the human body to maintain a passage and is covered with a PTFE film to prevent a lesion portion from intruding into a body stent, making it easier to perform a procedure along a curved lumen than the related arts.

Another objective of the present disclosure is to provide a covered stent in which there is displacement in hook parts and cross parts through a gap space formed between a body stent and a PTFE film so that the body stent is easily deformed to match the shape of a curved lumen depending on displacement of the hook parts and the cross parts, making it easier to perform a procedure along the curved lumen than the related arts.

To achieve the above objectives, according to the present disclosure, there is provided a covered stent that is easily positioned within a curved lumen and, to expand a lesion that causes stenosis or obstruction in a lumen in the body to maintain a passage of the lumen, the covered stent may include a cylindrical body stent in which a plurality of hook parts and a plurality of cross parts may be formed by weaving shape-memory alloy wires to be caught in a hook shape or crossing the wires so that a plurality of space parts may be formed among the wires, and expansion tube parts formed at opposite ends of the body stent and having a larger diameter than a diameter of the body stent, a first film made of a polytetrafluoroethylene (PTFE) material may be formed to cover an outer surface of the body stent, a second film made of a polytetrafluoroethylene (PTFE) material may be formed to cover outer surfaces of the expansion tube parts and outer surfaces of opposite end parts of the first film, which are adjacent to bent parts of the expansion tube parts, a third film made of a polytetrafluoroethylene (PTFE) material may be formed to cover inner surfaces of the expansion tube parts and inner surfaces of opposite end parts of the body stent, which are adjacent to the bent parts of the expansion tube parts, and extend in a longitudinal direction of the body stent longer than the second film, and the first, second, and third films may be thermally bonded, and a gap space may be formed between a portion of the first film, which is not bonded to the second and third films, and the body stent.

According to the first embodiment of the present disclosure, the PTFE first film covers the outer or inner surface of the body stent prevents a lesion portion from intruding into the body stent, and there is an effect of preventing the lesion portion from blocking the inside space of the body stent.

For this reason, the covered stent of the present disclosure expands a stenotic or occlusive lesion occurring in a lumen of the human body to maintain a passage of the lumen.

According to the first embodiment of the present disclosure, the covered stent has displacement at the hook parts and the cross parts through a gap space formed between a portion of the first film made of a PTFE material that is separated from the body stent and the body stent.

Therefore, there is an effect of ensuring flexibility of the body stent through the gap space.

For this reason, the covered stent of the present disclosure can be easily deformed to match the shape of a curved lumen by the displacement of the hook parts and the cross parts, and bending at a curved portion in the lumen is prevented, preventing broken wires or a broken stent due to the bending for long time.

According to the first embodiment of the present disclosure, since the first, second, and third films made of a PTFE material are bonded to seal a gap between the first, second, and third films, intruding of a lesion is prevented, so there are effects of maintaining a passage in the lumen and preventing contamination from occurring due to body fluids, bile, urine, and lodging of food particles passing through the lumen.

According to the first embodiment of the present disclosure, since ends of the first and fourth films made of a PTFE material are covered with the fifth silicone film to seal a gap among the first, fourth, and fifth films, intruding of a lesion portion is prevented, so there are effects of maintaining a passage in the lumen and preventing contamination from occurring due to body fluid, bile, urine, and lodging of food particles passing through the lumen.

According to the first embodiment of the present disclosure, since the first, second, and third films made of a PTFE material overlap with each other at the bent portion of the expansion tube part to increase the thickness, which can prevent easily bending the bent portion of the expansion tube part due to an external force such as movement of the human body.

In other words, the expansion tube part can continuously maintain an inserted state of the stent in the lumen.

For this reason, the covered stent of the present disclosure has an effect of not slipping in the lumen due to an external force such as movement of the human body.

According to the first embodiment of the present disclosure, since the first and fourth films made of a PTFE material and the fifth film made of a silicone material overlap with each other at the bent portion of the expansion tube part to increase the thickness, which can prevent easily bending the bent portion of the expansion tube part due to an external force such as movement of the human body.

In other words, the expansion tube part can continuously maintain an inserted state of the stent in the lumen.

For this reason, the covered stent of the present disclosure has an effect of not slipping in the lumen due to an external force such as movement of the human body.

According to the first embodiment of the present disclosure, the expansion tube parts have a strong recovery force that is to maintain a shape of the covered stent due to the fifth film made of a silicone material with high elasticity, so there is an effect that the expansion tube parts are not easily compressed due to an external force such as movement of the human body.

According to the first embodiment of the present disclosure, in the covered stent, as the cross parts are formed by between 20% and 80%, the hook parts are formed by between 80% and 20%, so there is an effect of preventing difficulty of the covered stent of the present disclosure in maintaining a curved state in the curved lumen due to a strong recovery force recovering the covered stent to a linear state, or preventing a radial force of the covered stent of the present disclosure from weakening to the point where the covered stent is pushed by a lesion.

For this reason, the covered stent of the first embodiment of the present disclosure can be easily deformed to match the curved lumen, not be pushed by the lesion, and maintain the passage in the lumen.

According to the second embodiment of the present disclosure, the sixth film made of a PTFE material covering the outer or inner surface of the body stent prevents a lesion portion from intruding into the body stent, and which can prevent the inside space of the body stent from being blocked by the lesion.

For this reason, the covered stent of the present disclosure expands a stenotic or occlusive lesion occurring in a lumen of the human body to maintain a passage of the lumen.

According to the second embodiment of the present disclosure, the covered stent has displacement at the hook parts and the cross parts through a gap space formed between a portion of the sixth film made of a PTFE material that is separated from the body stent and the body stent.

Therefore, there is an effect of ensuring flexibility of the body stent through the gap space.

For this reason, the covered stent of the present disclosure can be easily deformed to match the shape of a curved lumen by the displacement of the hook parts and the cross parts, and bending at a curved portion in the lumen is prevented, preventing broken wires or a broken stent due to the bending for long time.

According to the second embodiment of the present disclosure, since the sixth and seventh films made of a PTFE material are bonded to seal a gap between the sixth and seventh films, intruding of a lesion portion is prevented, so there are effects of maintaining a passage in the lumen and preventing contamination from occurring due to body fluids, bile, urine, and lodging of food particles passing through the lumen.

According to the second embodiment of the present disclosure, since the eighth film made of a silicone material and the ninth film made of a PTFE material are bonded to the sixth film made of a PTFE material to seal a gap between the sixth, eighth, and ninth films, intruding of a lesion portion is prevented, so there are effects of maintaining a passage in the lumen and preventing contamination from occurring due to body fluids, bile, urine, and lodging of food particles passing through the lumen.

According to the second embodiment of the present disclosure, since the highly elastic eighth film made of a silicone material is formed at bent portion of the expansion tube part and the opposite side portions of the body stent, the bent portion of the expansion tube part can be prevented from being easily bent due to an external force.

In other words, the expansion tube part can continuously maintain an inserted state of the stent in the lumen.

For this reason, the covered stent of the present disclosure has an effect of not slipping in the lumen due to an external force such as movement of the human body.

According to the second embodiment of the present disclosure, the expansion tube parts have a strong recovery force that is to maintain a shape of the covered stent due to the eighth film made of a silicone material with high elasticity, so there is an effect that the expansion tube parts are not easily compressed due to an external force such as movement of the body.

According to the second embodiment of the present disclosure, in the covered stent, as the cross parts are formed by between 20% and 80%, the hook parts are formed by between 80% and 20%, so there is an effect of preventing difficulty of the covered stent of the present disclosure in maintaining a curved state in the curved lumen due to a strong recovery force recovering the covered stent to a linear state, or preventing a radial force of the covered stent of the present disclosure from weakening to the point where the covered stent is pushed by a lesion.

For this reason, the covered stent of the second embodiment of the present disclosure can be easily deformed to match the curved lumen, not be pushed by the lesion, and maintain the passage in the lumen.

### BRIEF DESCRIPTION OF THE DRAWINGS

FIGS. 1 to 5 are a front view, an enlarged view, and a procedure state view of a conventional first stent,

FIGS. 6 to 8 are a front view, an enlarged view, and a procedure state view of a conventional second stent,

FIGS. 9 to 11 are a front view, an enlarged view, and a procedure state view of a conventional third stent that is covered with a PTFE film,

FIG. 12 is a picture partially taken with a bent state of a real conventional third stent produced from FIG. 11 partially,

FIG. 13 is a procedure state of a conventional third stent covered with a silicone film,

FIG. 14 is a picture taken with a bent state a real conventional third stent produced from FIG. 13,

FIG. 15 is a front view of a covered stent according to a first embodiment of the present disclosure,

FIGS. 16 to 19 are partial enlarged views of the covered stent according to the first embodiment of the present disclosure,

FIG. 20 is a sectional view of the covered stent according to the first embodiment of the present disclosure,

FIGS. 21 to 23 are forming process views of first, second, and third films of the covered stent according to the first embodiment of the present disclosure,

FIG. 24 is a picture taken with a bent state of a real covered stent according to the first embodiment of the present disclosure,

FIGS. 25 to 27 are procedure state views showing a procedure of the covered stent according to the first embodiment of the present disclosure on a curved lumen,

FIGS. 28 to 30 are partially enlarged views of a procedure state of the covered stent according to the first embodiment of the present disclosure,

FIG. 31 is a procedure state view taken with a procedure of the covered stent according to the first embodiment of the present disclosure on a linear lumen,

FIG. 32 is a front view of the covered stent according to a first modification of the first embodiment of the present disclosure,

FIG. 33 is a partially enlarged view of the covered stent according to the first modification of the first embodiment of the present disclosure,

FIGS. 34 to 36 are forming process views of the first, second, and third films of the covered stent according to the first modification of the first embodiment of the present disclosure,

FIG. 37 is a partially enlarged view showing a procedure state of the covered stent according to the first modification of the first embodiment of the present disclosure,

FIG. 38 is a front view of the covered stent according to a second modification of the first embodiment of the present disclosure,

FIG. 39 is a partially enlarged view of the covered stent according to the second modification of the first embodiment of the present disclosure,

FIGS. 40 and 41 are forming process views of first, fourth, and fifth films of the covered stent according to the second modification of the first embodiment of the present disclosure,

FIG. 42 is a procedure state view showing a procedure of the covered stent according to the second modification of the first embodiment of the present disclosure on a curved lumen,

FIG. 43 is a front view of the covered stent according to a third modification of the first embodiment of the present disclosure,

FIGS. 44 and 45 are forming process views of first, fourth, and fifth films of the covered stent according to the third modification of the first embodiment of the present disclosure,

FIG. 46 is a partially enlarged view showing a procedure state of the covered stent according to the third modification of the first embodiment of the present disclosure,

FIG. 47 is a front view of a covered stent according to a second embodiment of the present disclosure,

FIGS. 48 to 50 are partial enlarged views of the covered stent according to the second embodiment of the present disclosure,

FIG. 51 is a sectional view of a covered stent according to a second embodiment of the present disclosure,

FIGS. 52 and 53 are forming process views of sixth and seventh films of the covered stent according to the second embodiment of the present disclosure,

FIGS. 54 to 56 are procedure state views showing a procedure of the covered stent according to the second embodiment of the present disclosure on a curved lumen,

FIGS. 57 to 58 are partially enlarged views of a procedure state of the covered stent according to the second embodiment of the present disclosure,

FIG. 59 is a front view of the covered stent according to a first modification of the second embodiment of the present disclosure,

FIG. 60 is a partially enlarged view of the covered stent according to the first modification of the second embodiment of the present disclosure,

FIGS. 61 and 62 are forming process views of the sixth and seventh films of the covered stent according to the first modification of the second embodiment of the present disclosure,

FIG. 63 is a partially enlarged view showing a procedure state of the covered stent according to the first modification of the second embodiment of the present disclosure,

FIG. 64 is a front view of the covered stent according to a second modification of the second embodiment of the present disclosure,

FIG. 65 is a partially enlarged view of the covered stent according to the second modification of the second embodiment of the present disclosure,

FIGS. 66 and 67 are forming process views of the sixth and seventh films of the covered stent according to the second modification of the second embodiment of the present disclosure,

FIG. 68 is a procedure state view showing a procedure of the covered stent according to the second modification of the second embodiment of the present disclosure on a curved lumen,

FIG. 69 is a front view of the covered stent according to a third modification of the second embodiment of the present disclosure,

FIG. 70 is a partially enlarged view of the covered stent according to the third modification of the second embodiment of the present disclosure,

FIGS. 71 and 72 are forming process views of the sixth and seventh films of the covered stent according to the third modification of the second embodiment of the present disclosure, and

FIG. 73 is a partially enlarged view showing a procedure state of the covered stent according to the third modification of the second embodiment of the present disclosure.

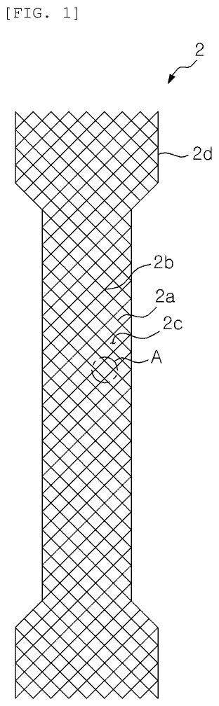

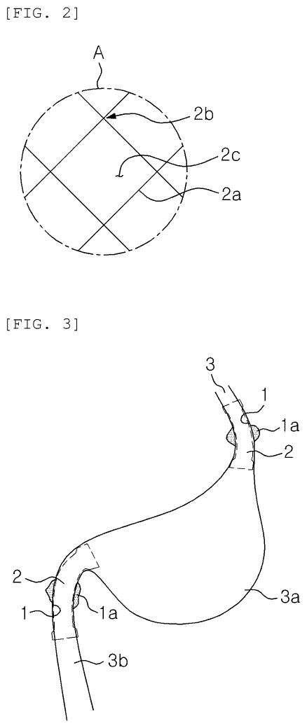

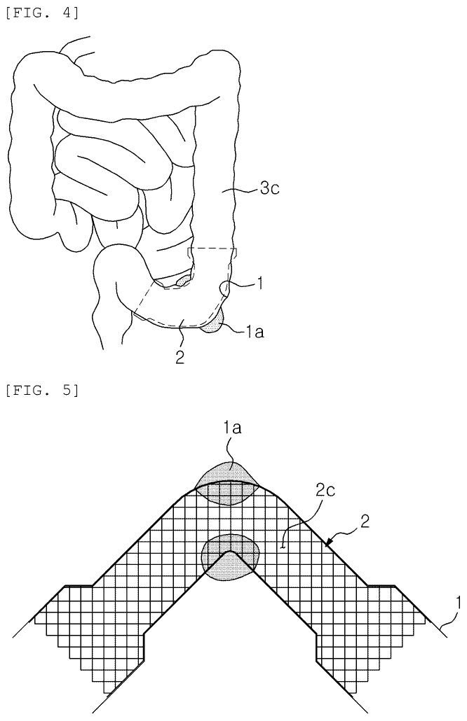

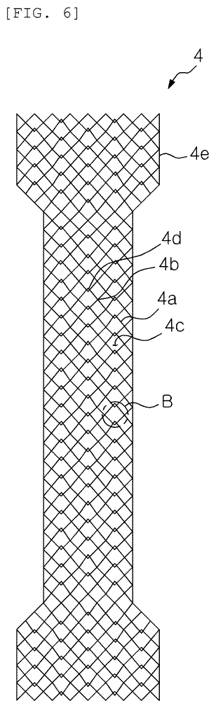

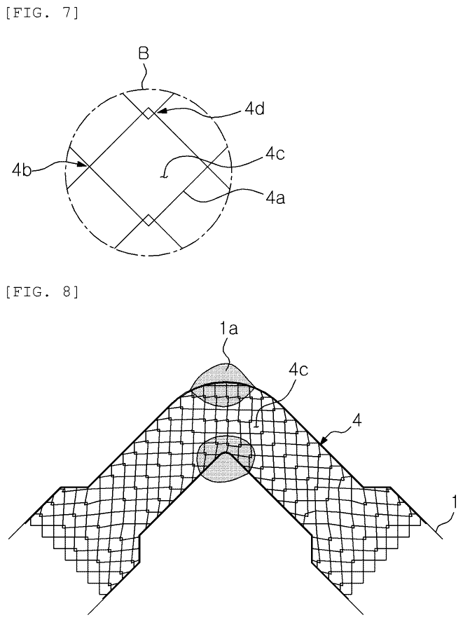

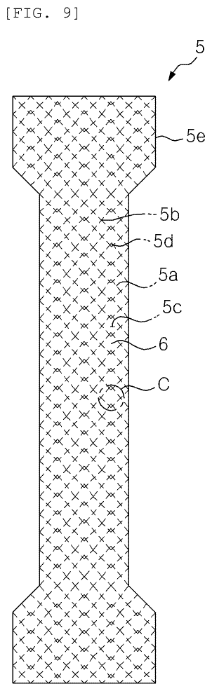

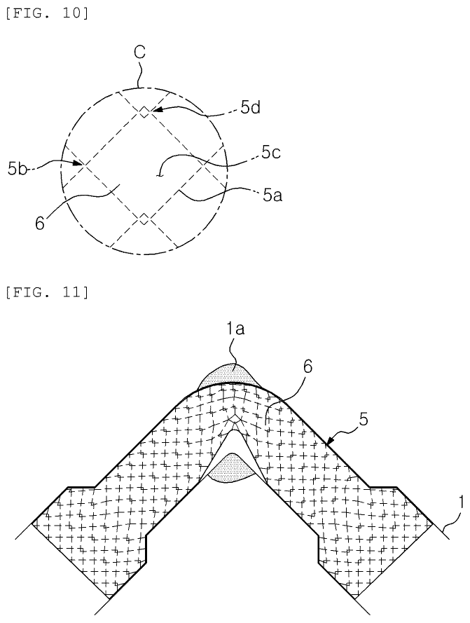

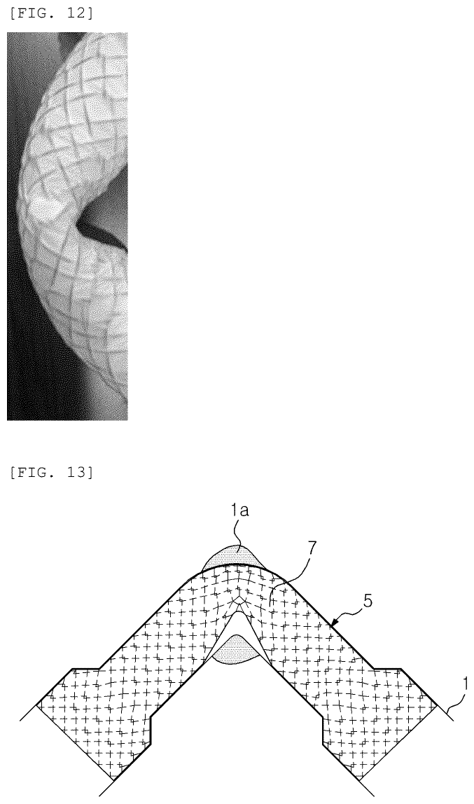

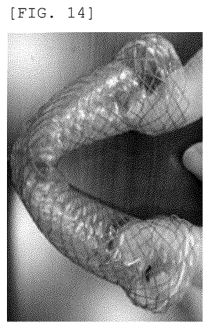

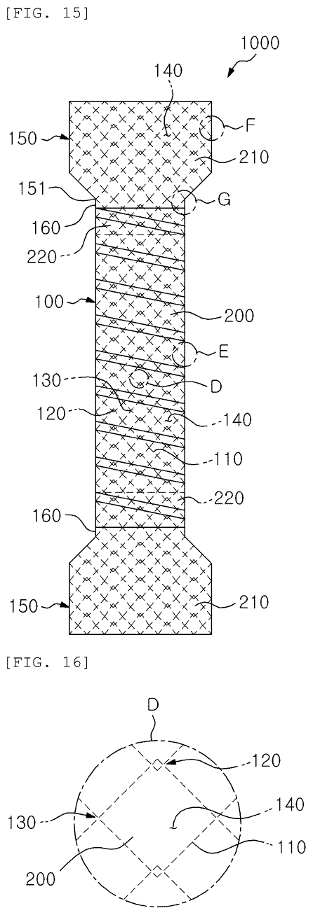

### DETAILED DESCRIPTION OF THE DISCLOSURE

Accordingly, exemplary embodiments of the present disclosure will be described in detail will be described with reference to the accompanying drawings as follows.

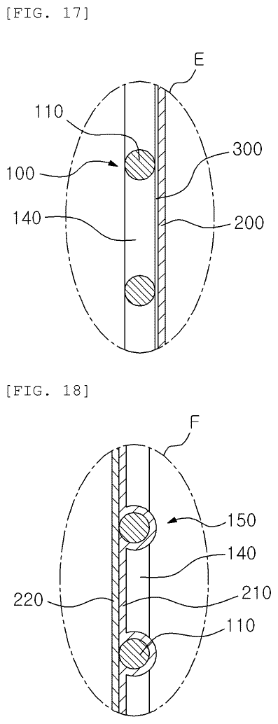

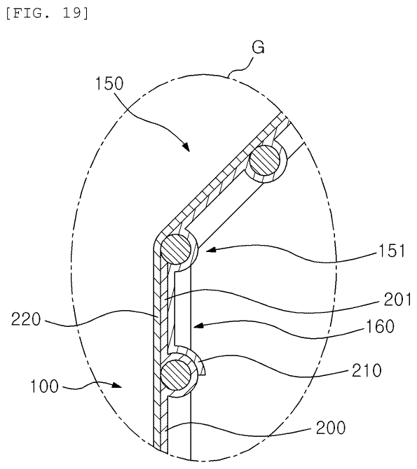

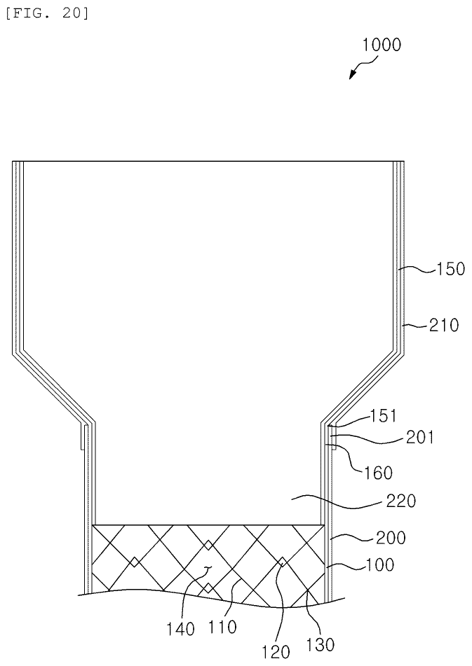

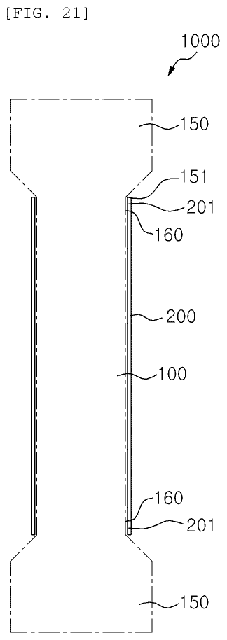

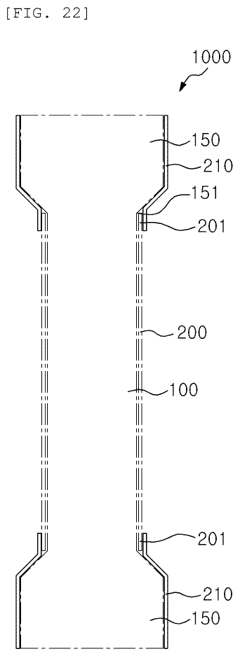

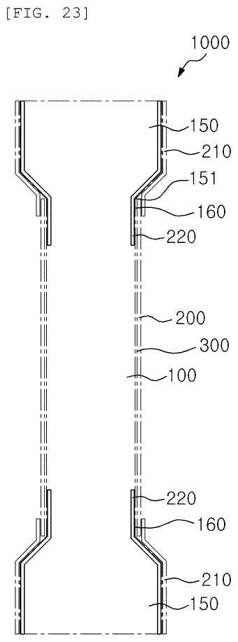

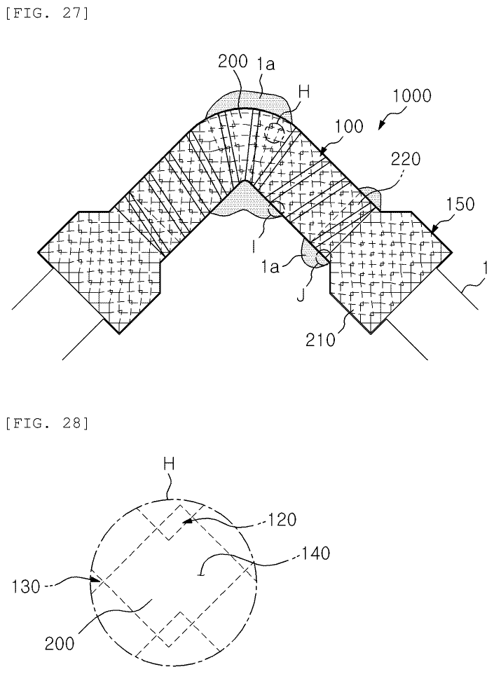

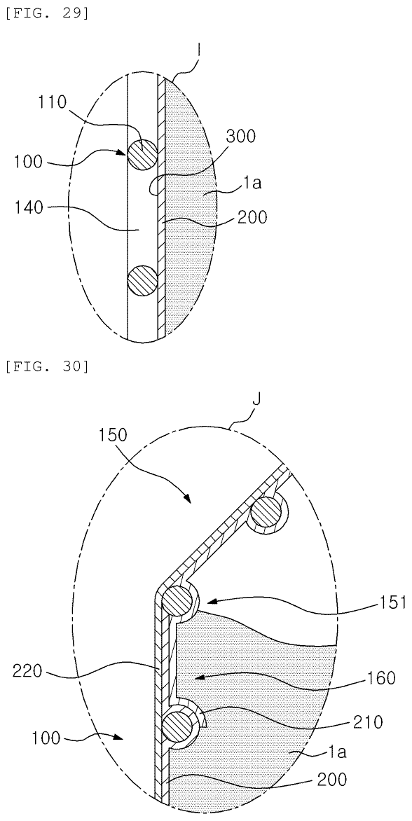

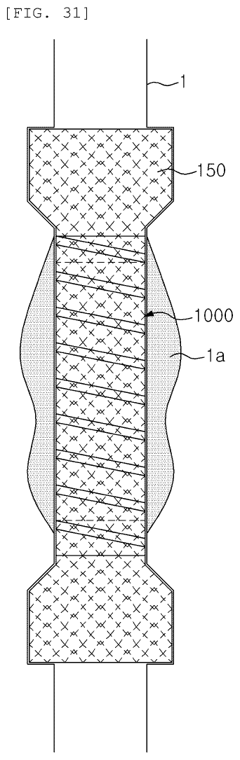

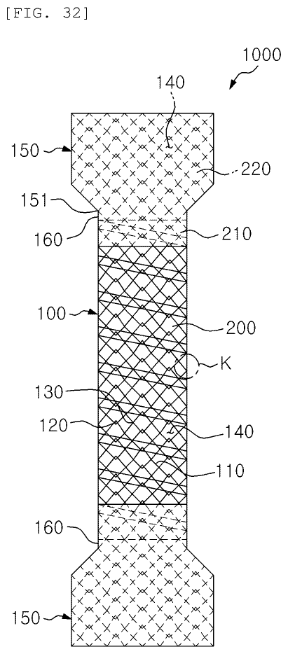

As shown in FIGS. 15 to 31, according to a first embodiment of the present disclosure, a covered stent 1000 is used to be inserted into a lumen 1 in the human body, such as biliary tract, esophagus, ureters, or large intestine of the human body, with a stent delivery system such as a catheter, to expand a stenotic or occlusive lesion portion 1a occurring in the lumen 1 to maintain a passage in the lumen 1.

The covered stent 1000 includes a body stent 100, expansion tube parts 150, first, second, and third films 200, 210, and 220 made of a polytetrafluoroethylene (PTFE) material, and a gap space 300.

The body stent 100 is shaped in a cylinder in which shape-memory alloy wires 110 are caught by each other in a hook shape or cross to form a plurality of hook parts 120 and a plurality of cross parts 130 so that a plurality of space parts 140 is formed among the wires 110.

The expansion tube parts 150 are formed at opposite ends of the body stent 100 and each has a larger diameter than the body stent 100.

At this point, bent parts 151 are formed at ends of the expansion tube parts 150 to connect the expansion tube parts 150 and the body stent 100 to each other.

The first film 200 made of a PTFE material is formed to cover an outer surface of the body stent 100.

At this point, the first film 200 may be made of a PTFE tape and have a spiral shape or a ring shape on the outer surface of the body stent 100 so that overlap portions are formed by overlapping each other.

The second film 210 made of a PTFE material is formed to cover outer surfaces of the expansion tube parts 150 and outer surfaces of opposite end parts 201 of the first film 200, the opposite ends being adjacent to the bent parts 151 of the expansion tube parts 150.

At this point, the second film 210 may be made of PTFE tape and have a spiral shape or a ring shape on the outer surfaces of the expansion tube parts 150 and the outer surfaces of the opposite end parts 201 of the first film 200 so that overlap portions are formed by overlapping each other.

The third film 220 made of a PTFE tape is formed to cover inner surfaces of the expansion tube parts 150 and inner surfaces of opposite end parts 160 of the body stent 100, which are adjacent to the bent parts 151 of the expansion tube parts 150, and extend in a longitudinal direction of the body stent 100 longer than the second film 210.

At this point, so that the third film 220 may be made of a PTFE tape and have a spiral shape or a ring shape on the inner surfaces of the expansion tube parts 150, the inner surfaces of the opposite end parts 160 of the body stent 100, and the opposite portions of the inner surface of the body stent 100, wherein the opposite portions are adjacent to the inner surfaces of the opposite end parts 160, so that overlap portions are formed by overlapping each other.

The first, second, and third films 200, 210, and 220 may be thermally bonded.

At this point, the hook parts 120 and the cross parts 130 located at the bonded first, second, and third films 200, 210, and 220 are fixed to the first, second, and third films 200, 210, and 220, so the hook parts 120 and the cross parts 130 are not free to be displaced.

Furthermore, the first, second, and third films 200, 210, and 220 prevent the space parts 140 from being exposed outwards.

Furthermore, in the bent parts 151 of the expansion tube parts 150, the overlap portions of the first, second, and third films 200, 210, and 220 are thicker than non-overlapping portions of the first, second, and third films 200, 210, and 220.

Furthermore, gaps between the first, second, and third films 200, 210, and 220 are sealed as the first, second, and third films 200, 210, and 220 are bonded with each other.

The gap space 300 is formed between a portion of the first film 200, which is not bonded to the second and third films 210 and 220, and the body stent 100.

In other words, as the portion not bonded to the second and third films 210 and 220 comes loose, the gap space 300 is formed between a portion of the first film 200 not bonded to the second and third films 210 and 220 and the outer surface of the body stent 100.

At this point, the hook parts 120 and the cross parts 130 located in the gap space 300 are not fixed to the first film 200 due to the gap space 300 and are free to be displaced.

The cross parts 130 are formed between 20% and 80% of the covered stent 1000, so the hook parts 120 are formed between 80% and 20% of the covered stent 1000.

As a reason, as the cross parts 130 are formed equal to or higher than 80% of the covered stent 1000, i.e., the body stent 100 and the expansion tube parts 150, when the hook parts 120 are formed less than or equal to 20% of the covered stent 1000, i.e., the body stent 100 and the expansion tube parts 150, the covered stent 1000 has a strong recovery force provided by the cross parts 130 formed 80% or more and maintain a linear shape, so it is difficult for the covered stent 1000 to remain covered.

Furthermore, as the cross parts 130 are formed equal to or higher than 80%, when the covered stent 1000 with the hook parts 120 formed less than or equal to 20% is positioned within a curved lumen 1, the covered stent 1000 has difficulty in maintaining a curved state due to a strong recovery force that recovers the covered stent to a linear state.

Furthermore, a portion of the body stent 100 located on a covered portion of the lumen 1 is subjected to stress for a long time, and there is a risk that the wires 110 will break due to the prolonged stress.

Furthermore, as the cross parts 130 are formed by less than or equal to 20% of the covered stent 1000, when the hook parts 120 are formed by equal to or higher than 80% of the covered stent 1000, the covered stent 1000 easily maintains a curved state because a force to recover the covered stent to a linear state is weakened due to the hook parts 120 formed by 80% or more, but a radial force is weakened.

Accordingly, when the covered stent 1000 in which the cross parts 130 are formed by 20% or less and the hook parts 120 are formed 80% or more is positioned within the curved lumen 1, the covered stent 1000 is easily deformed to fit the curved lumen 1, but the covered stent 1000 is pressed by the lesion portion 1a due to a weakened radial force, and the passage of the lumen 1 is narrowed.

Because of this, the cross parts 130 are formed between 20% and 80% of the covered stent 1000, so the hook parts 120 are formed between 80% and 20% of the covered stent 1000.

Ideally, the cross parts 130 and the hook parts 120 are each formed by 50% of the covered stent 1000.

The expansion tube parts 150 may include a pull string that is used to remove the covered stent 1000.

According to the first embodiment of the present disclosure, operation of the covered stent 1000 will be described as follows.

As shown in FIGS. 15 to 31, the covered stent 1000 is positioned within the curved lumen 1 by an endoscope that checks a location where the procedure of the covered stent is performed and a stent delivery system that delivers the stent to the location of the procedure.

In other words, as shown in FIGS. 25 to 27, the covered stent 1000 is positioned within a portion of the curved lumen 1 where the esophagus 3 and the stomach 3a are connected to each other, a portion of the curved lumen 1 where the stomach 3a and the duodenum 3b are connected to each other, and a portion of the curved lumen 1 where the large intestine 3c is curved.

Thereafter, the hook parts 120 and the cross parts 130 located in the gap space 300 are not fixed to the first film 200 by the gap space 300, so displacement occurs in the hook parts 120 and the cross parts 130 in correspondence to the curved lumen 1.

Furthermore, the space parts 140 are also deformed due to the displacement of the hook parts 120 and the cross parts 130.

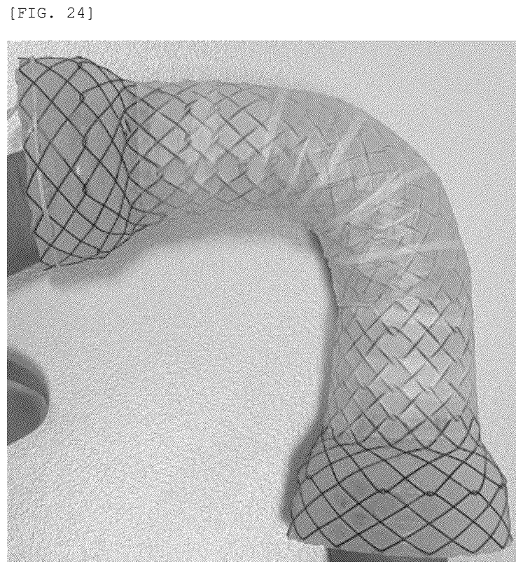

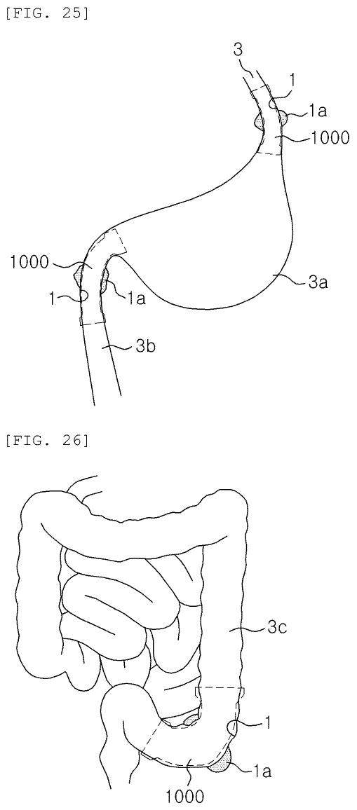

For this reason, as shown in FIGS. 24 to 27, the covered stent 1000 is easily deformed in a curved shape that fits the curved lumen 1 and expands the lesion portion 1a, thereby maintaining the passage of the lumen 1.

Furthermore, the covered stent 1000 is easily deformed in a curved shape to fit the curved lumen 1 without bending at the curved portion of the lumen 1.

Furthermore, the expansion tube parts 150 of the covered stent 1000 are inserted in and caught by the lumen 1.

Furthermore, in the bent parts 151 of the expansion tube parts 150, the overlap portions of the first, second, and third films 200, 210, and 220 have the thickness thicker than the thickness of the non-overlap portions of the first, second, and third films 200, 210, and 220, which prevents the bent parts 151 of the expansion tube parts 150 from being easily bent due to external force such as movement of the human body.

Furthermore, the first, second, and third films 200, 210, and 220 made of a PTFE material may prevent the lesion portion 1a from intruding into the covered stent 1000.

Furthermore, a gap between the first, second, and third films 200, 210, and 220 is sealed when the first, second, and third films 200, 210, and 220, thereby preventing the lesion portion 1a from intruding into the covered stent 1000.

As shown in FIG. 31, the covered stent 1000 is positioned within a linear lumen 1 by an endoscope that checks a location of the procedure and a stent delivery system that delivers the stent to the location of the procedure.

Furthermore, when the covered stent 1000 expands the lesion portion 1a in the linear lumen 1, the expansion tube parts 150 are inserted into and caught by the lumen 1.

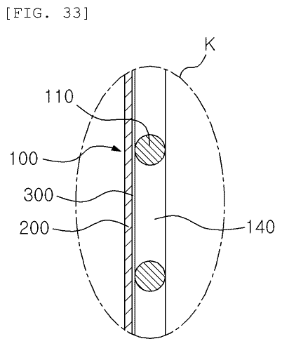

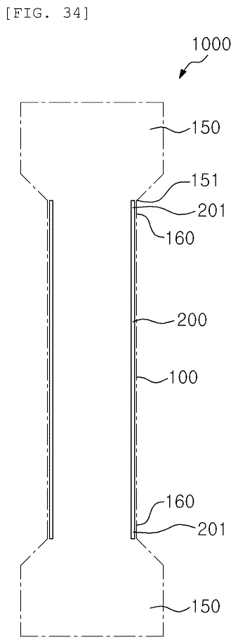

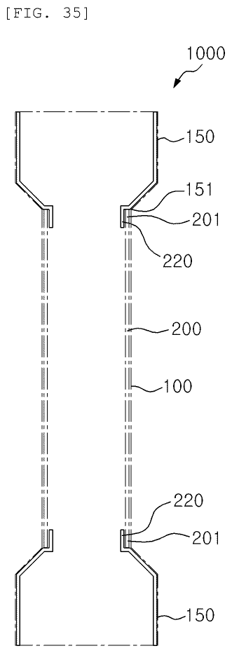

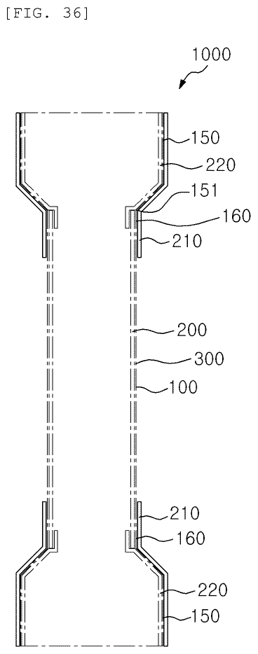

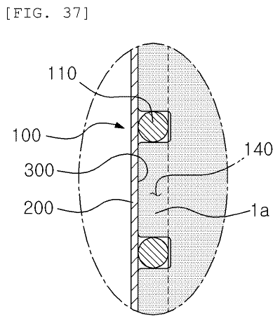

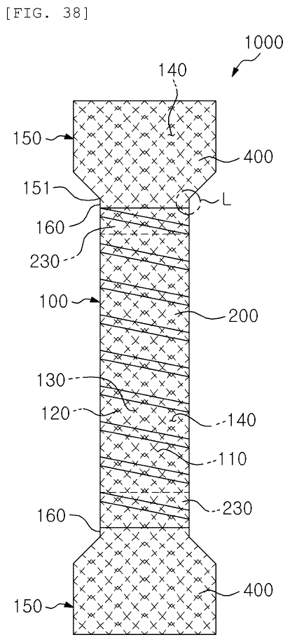

As shown in FIGS. 32 to 37, according to the first modification of the first embodiment of the present disclosure, the first film 200 of the covered stent 1000 may be formed to cover the inner surface of the body stent 100.

The third film 220 is formed to cover the inner surfaces of the expansion tube parts 150 and the inner surfaces of the opposite end parts 201 of the first film 200.

The second film 210 is formed to cover the outer surfaces of the expansion tube parts 150 and the outer surfaces of the opposite end parts 160 of the body stent 100 and extends in the longitudinal direction of the body stent 100 longer than the third film 220.

Therefore, when the first, second, and third films 200, 210, and 220 are thermally bonded, the gap space 300 may be formed between a portion of the first film 200 to which the second and third films 210 and 220 and the body stent 100.

At this point, the gap space 300 is formed between a portion of the first film 200, which is not bonded to the second and third films 210 and 220, and the inner surface of the body stent 100.

Furthermore, the space parts 140 located in the gap space 300 are exposed outwards.

Therefore, when the covered stent 1000 is positioned within the curved lumen 1, the lesion portion 1a intrudes into the space parts 140 located in the gap space 300.

For this reason, the space parts 140 of the covered stent 1000 are caught by the lesion portion 1a intruding into the stent.

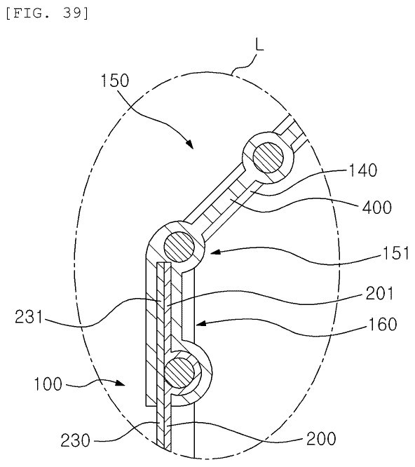

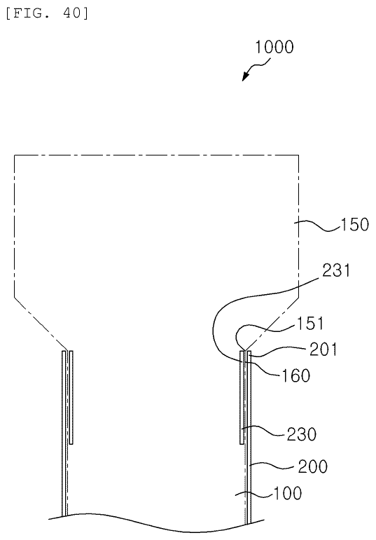

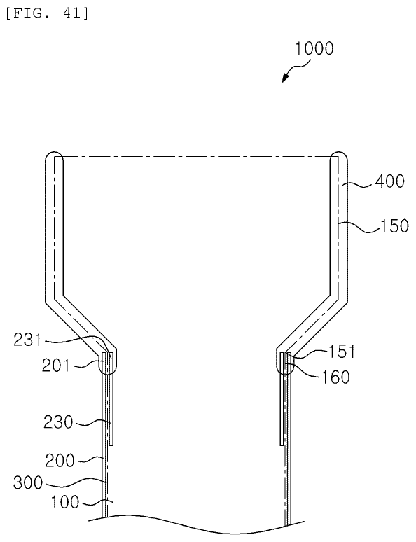

As shown in FIGS. 38 to 42, the second modification of the first embodiment of the present disclosure, the covered stent 1000 excludes the second and third films 210 and 220, and includes a fourth film 230 made of a PTFE material and a fifth film 400 made of a silicone material.

The fourth film 230 made of a PTFE material covers the inner surfaces of the opposite end parts 160 of the body stent 100 and the opposite portions of the inner surface of the body stent 100, which are adjacent to the opposite end parts, and are thermally bonded to the first film 200.

At this point, the fourth film 230 may be made of a PTFE tape and have a spiral shape or a ring shape on the inner surfaces of the opposite end parts 160 of the body stent 100 and the opposite portions of the inner surface of the body stent 100, which are adjacent to the opposite end portions, so that overlap portions are formed by overlapping each other.

Furthermore, the first and fourth films 200 and 230 prevent the space parts 140 from being exposed outwards.

Furthermore, the hook parts 120 and the cross parts 130 located at the bonded first and fourth films 200 and 230 are fixed to the first and fourth films 200 and 230, so the hook parts 120 and the cross parts 130 are not free to be displaced.

The gap space 300 is formed between a portion of the first film 200, which is not bonded to the fourth film 230, and the body stent 100.

In other words, as the portion of the first film 200 not bonded to the fourth film 230 comes loose, the gap space 300 is formed between a portion of the first film 200 not bonded to the fourth film 230 and the outer surface of the body stent 100.

At this point, the hook parts 120 and the cross parts 130 located in the gap space 300 are not fixed to the first film 200 due to the gap space 300 and are free to be displaced.

The fifth film 400 covers the expansion tube parts 150, one end part 231 of the fourth film 230 adjacent to the bent parts 151 of the expansion tube parts 150, and the opposite end parts 201 of the first film 200 by a silicone dipping method.

At this point, the hook parts 120 of the expansion tube parts 150 and the cross parts 130 are not free to change as fixed to the fifth film 400, and the space parts 140 of the expansion tube parts 150 are not exposed outwards by the fifth film 400.

Furthermore, in the bent parts 151 of the expansion tube parts 150, the overlap portions of the first, fourth, and fifth films 200, 230, and 400 are thicker than non-overlapping portions of the first, fourth, and fifth films 200, 230, and 400.

Furthermore, a gap between the first, fourth, and fifth films 200, 230, and 400 is sealed as ends of 201 and 231 of the bonded first and fourth films 200 and 230 are filled with the fifth film 400 made of a silicone material.

Therefore, when the covered stent 1000 is positioned within the curved lumen 1, the hook parts 120 and the cross parts 130 located in the gap space 300 are not fixed to the first film 200 by the gap space 300, so displacement occurs in the hook parts 120 and the cross parts 130 in correspondence to the curved lumen 1.

Furthermore, the space parts 140 are also deformed due to the displacement of the hook parts 120 and the cross parts 130.

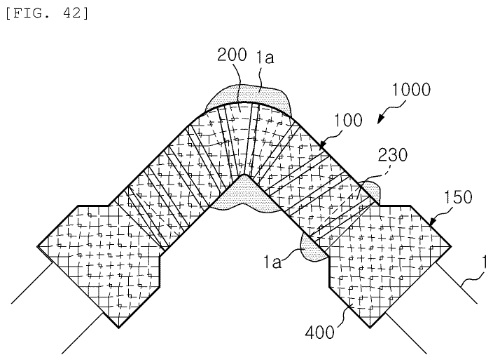

For this reason, as shown in FIG. 42, the covered stent 1000 is easily deformed in a curved shape that fits the curved lumen 1 and expands the lesion portion 1a, thereby maintaining the passage of the lumen 1.

Furthermore, the covered stent 1000 is easily deformed in a curved shape to fit the curved lumen 1 without bending at the curved portion of the lumen 1.

Furthermore, the expansion tube parts 150 of the covered stent 1000 are inserted in and caught by the lumen 1.

Furthermore, the expansion tube parts 150 are covered with the fifth film 400 made of a highly elastic silicone material and have strong resilience to maintain original forms thereof, so the expansion tube parts 150 are not easily compressed by external force such as movement of the human body.

Furthermore, in the bent parts 151 of the expansion tube parts 150, the overlap portions of the first, fourth, and fifth films 200, 230, and 400 have the thickness thicker than the thickness of the non-overlap portions of the first, fourth, and fifth films 200, 230, and 400, which prevents the bent parts 151 of the expansion tube parts 150 from being easily bent due to external force such as movement of the human body.

Furthermore, the first and fourth films 200 and 230 made of a PTFE material and the fifth film 400 made of a silicone material may prevent the lesion portion 1a from intruding into the covered stent 1000.

Furthermore, a gap between the first, fourth, and fifth films 200, 230, and 400 is sealed when the end portions 201 and 231 of the bonded first and fourth films 200 and 230 are covered with the fifth film 400 made of a silicone material, which prevents the lesion portion 1a from intruding into the covered stent 1000.

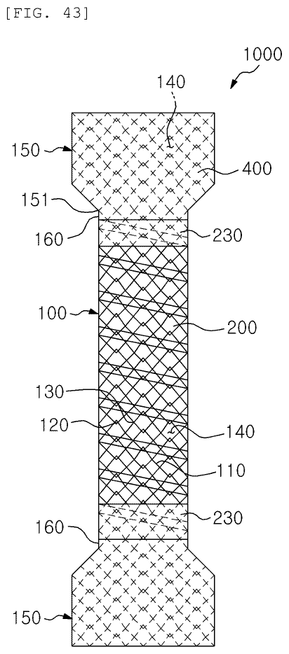

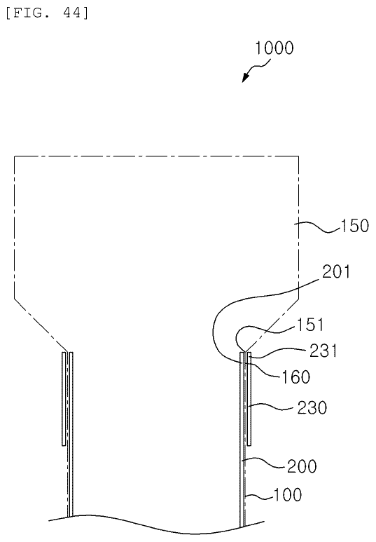

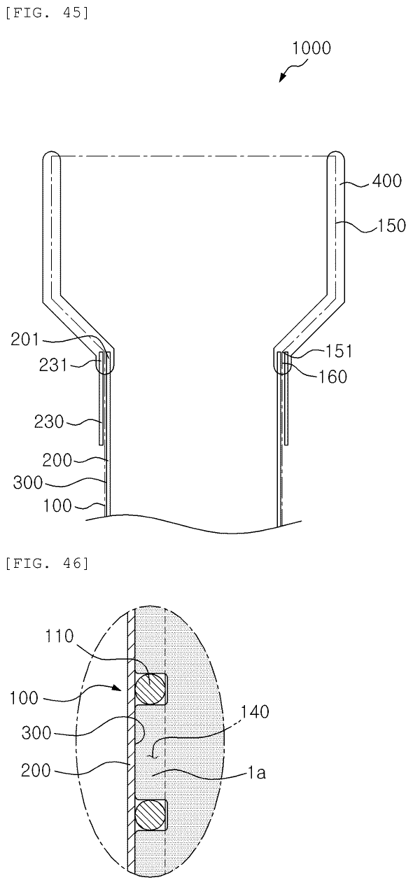

As shown in FIGS. 43 to 46, according to the third modification of the first embodiment of the present disclosure, the covered stent 1000 is configured to be the same as the second modification with different positions where the first and fourth films 200 and 230 of the second modification are installed.

The first film 200 may be formed to cover the inner surface of the body stent 100.

The fourth film 230 is formed to cover the outer surfaces of the opposite end parts 160 of the body stent 100 and the opposite portions of the outer surface of the body stent 100 that are adjacent thereto.

Therefore, when the first and fourth films 200 and 230 are thermally bonded, the gap space 300 is formed between a portion of the first film 200 not bonded to the fourth film 230 and the body stent 100.

At this point, the gap space 300 is formed between a portion of the first film 200, which is not bonded to the fourth film 230, and the inner surface of the body stent 100.

Furthermore, the space parts 140 located in the gap space 300 are exposed outwards.

The fifth film 400 covers the expansion tube parts 150, one end part 231 of the fourth film 230, and the opposite end parts 201 of the first film 200, by a silicone dipping method.

Therefore, when the covered stent 1000 is positioned within the curved lumen 1, the lesion portion 1a intrudes into the space parts 140 located in the gap space 300.

For this reason, the space parts 140 of the covered stent 1000 are caught by the lesion portion 1a intruding into the stent.

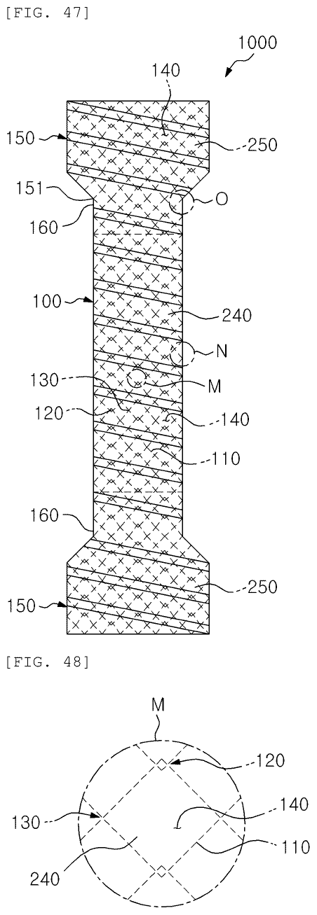

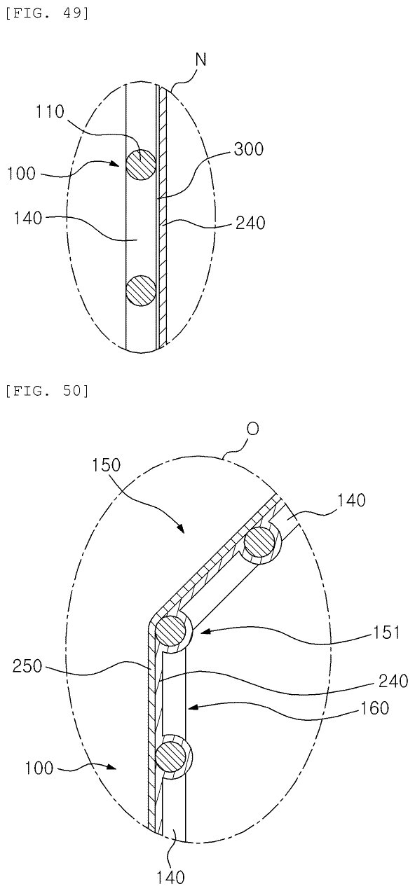

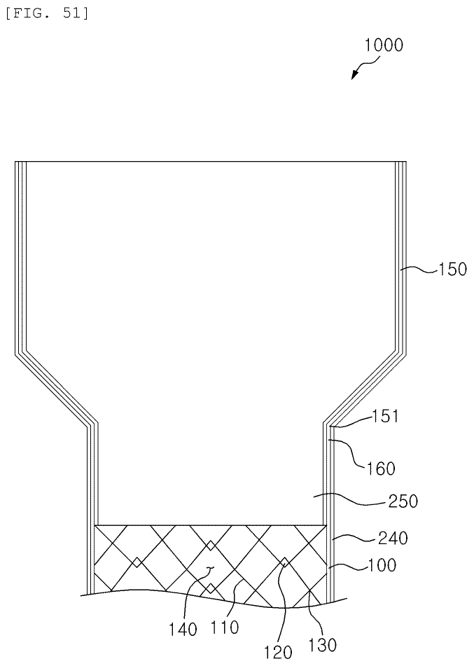

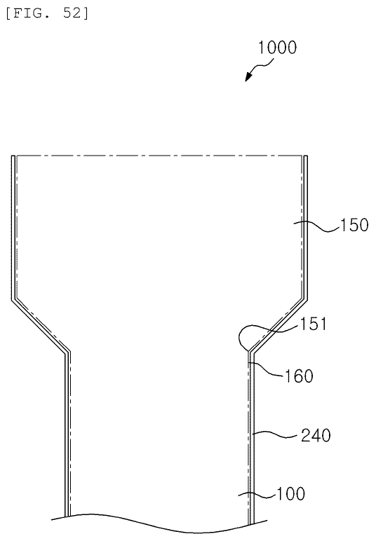

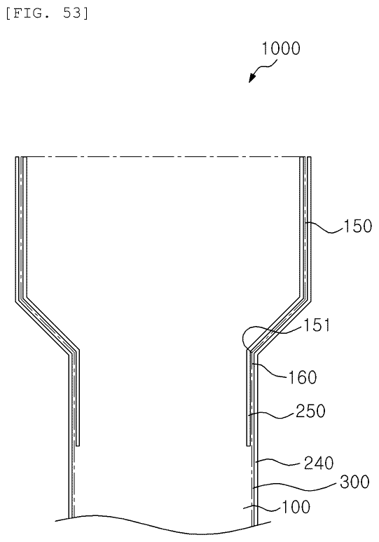

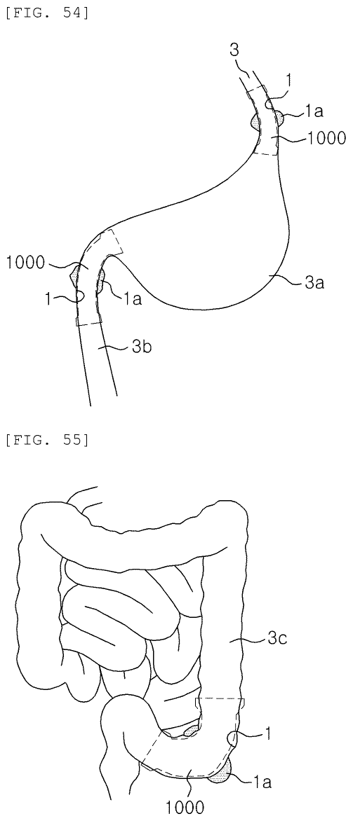

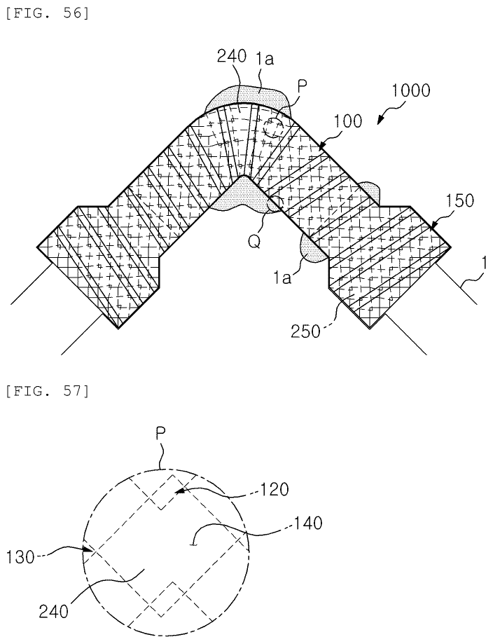

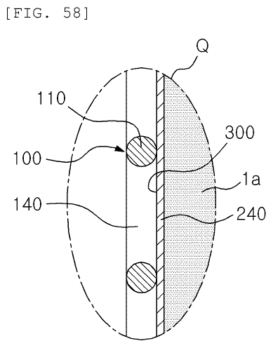

As shown in FIGS. 47 to 58, according to a second embodiment of the present disclosure, the covered stent 1000 is used to be inserted into a lumen 1 in the human body, such as the biliary tract, the esophagus, ureters, or large intestine of the human body, with a stent delivery system such as a catheter, to expand a stenotic or occlusive lesion portion 1a occurring in the lumen 1 to maintain a path in the lumen 1.

The covered stent 1000 includes the body stent 100, the expansion tube parts 150, sixth and seventh films 240 and 250 made of a polytetrafluoroethylene (PTFE) material, and the gap space 300.

The body stent 100 has a cylindrical shape in which shape-memory alloy wires 110 are caught by each other in a hook shape or cross to form a plurality of hook parts 120 and a plurality of cross parts 130 so that a plurality of space parts 140 is formed among the wires 110.

The expansion tube parts 150 are formed at opposite ends of the body stent 100 and each has a larger diameter than the body stent 100.

At this point, bent parts 151 are formed at ends of the expansion tube parts 150 to connect the expansion tube parts 150 and the body stent 100 to each other.

The sixth film 240 made of a PTFE material is formed to cover the outer surface of the body stent 100 and the outer surfaces of the expansion tube parts 150.

At this point, the sixth film 240 may be made of a PTFE tape and have a spiral shape or a ring shape on the outer surface of the body stent 100 and the outer surfaces of the expansion tube parts 150 so that overlap portions are formed by overlapping each other.

The seventh film 250 made of a PTFE material is formed to cover the inner surfaces of the expansion tube parts 150, the inner surfaces of the opposite end parts 160 of the body stent 100 adjacent to the bent parts 151 of the expansion tube parts 150, and the opposite portions of the inner surface of the body stent 100 adjacent thereto.

At this point, the seventh film 250 may be made of a PTFE tape and have a spiral shape or a ring shape on the inner surfaces of the expansion tube parts 150, the inner surfaces of the opposite end parts 160 of the body stent 100, and the opposite portions of the inner surface of the body stent 100, which are adjacent to the opposite end portions, so that overlap portions are formed by overlapping each other.

The sixth and seventh films 240 and 250 are bonded.

At this point, the hook parts 120 and the cross parts 130 located at the bonded the sixth and seventh films 240 and 250 are fixed to the sixth and seventh films 240 and 250, so the hook parts 120 and the cross parts 130 are not free to be displaced.

Furthermore, the sixth and seventh films 240 and 250 prevent the space parts 140 from being exposed outwards.

Furthermore, a gap between the sixth and seventh films 240 and 250 is sealed when the sixth and seventh films 240 and 250 are bonded.

The gap space 300 is formed between a portion of the sixth film 240, which is not bonded to the seventh film 250, and the body stent 100.

In other words, as the portion of the sixth film 240 not bonded to the seventh film 250 comes loose, the gap space 300 is formed between a portion of the sixth film 240 not bonded to the seventh film 250 and the outer surface of the body stent 100.

At this point, the hook parts 120 and the cross parts 130 located in the gap space 300 are not fixed to the fourth film 240 due to the gap space 300 and are free to be displaced.

The cross parts 130 are formed between 20% and 80% of the covered stent 1000, so the hook parts 120 are formed between 80% and 20% of the covered stent 1000.

As a reason, as the cross parts 130 are formed equal to or higher than 80% of the covered stent 1000, i.e., the body stent 100 and the expansion tube parts 150, when the hook parts 120 are formed less than or equal to 20% of the covered stent 1000, i.e., the body stent 100 and the expansion tube parts 150, the covered stent 1000 has a strong recovery force provided by the cross parts 130 formed 80% or more and maintain a linear shape, so it is difficult for the covered stent 1000 to remain covered.

Furthermore, as the cross parts 130 are formed equal to or higher than 80%, when the covered stent 1000 with the hook parts 120 formed less than or equal to 20% is positioned within a curved lumen 1, the covered stent 1000 has difficulty in maintaining a curved state due to a strong recovery force that recovers the covered stent to a linear state.

Furthermore, a portion of the body stent 100 located on a covered portion of the lumen 1 is subjected to stress for a long time, and there is a risk that the wires 110 will break due to the prolonged stress.

Furthermore, as the cross parts 130 are formed by less than or equal to 20% of the covered stent 1000, when the hook parts 120 are formed by equal to or higher than 80% of the covered stent 1000, the covered stent 1000 easily maintains a curved state because a force to recover the covered stent to a linear state is weakened due to the hook parts 120 formed by 80% or more, but a radial force is weakened.

Accordingly, when the covered stent 1000 in which the cross parts 130 are formed by 20% or less and the hook parts 120 are formed 80% or more is positioned within the curved lumen 1, the covered stent 1000 is easily deformed to fit the curved lumen 1, but the covered stent 1000 is pressed by the lesion portion 1a due to a weakened radial force, and the passage of the lumen 1 is narrowed.

Because of this, the cross parts 130 are formed between 20% and 80% of the covered stent 1000, so the hook parts 120 are formed between 80% and 20% of the covered stent 1000.

Ideally, the cross parts 130 and the hook parts 120 are each formed by 50% of the covered stent 1000.

The expansion tube parts 150 may include a pull string that is used to remove the covered stent 1000.

According to the second embodiment of the present disclosure, operation of the covered stent 1000 will be described as follows.

As shown in FIGS. 47 to 58, the covered stent 1000 is positioned within the curved lumen 1 by an endoscope that checks a location of the procedure and a stent delivery system that delivers the stent to the location of the procedure.

In other words, as shown in FIGS. 54 to 56, the covered stent 1000 is positioned within a portion of the curved lumen 1 where the esophagus 3 and the stomach 3a are connected to each other, a portion of the curved lumen 1 where the stomach 3a and the duodenum 3b are connected to each other, and a portion of the curved lumen 1 where the large intestine 3c is curved.

Thereafter, the hook parts 120 and the cross parts 130 located in the gap space 300 are not fixed to the sixth film 240 by the gap space 300, so displacement occurs in the hook parts 120 and the cross parts 130 in correspondence to the curved lumen 1.

Furthermore, the space parts 140 are also deformed due to the displacement of the hook parts 120 and the cross parts 130.

For this reason, as shown in FIGS. 54 to 56, the covered stent 1000 is easily deformed in a curved shape that fits the curved lumen 1 and expands the lesion portion 1a, thereby maintaining the passage of the lumen 1.

Furthermore, the covered stent 1000 is easily deformed in a curved shape to fit the curved lumen 1 without bending at the curved portion of the lumen 1.

Furthermore, the expansion tube parts 150 of the covered stent 1000 are inserted in and caught by the lumen 1.

Furthermore, the sixth and seventh films 240 and 250 made of a PTFE material may prevent the lesion portion 1a from intruding into the covered stent 1000.

Furthermore, a gap between the sixth and seventh films 240 and 250 is sealed when the sixth and seventh films 240 and 250 are bonded, thereby preventing the lesion portion 1a from intruding into the covered stent 1000.

Meanwhile, the covered stent 1000 may be positioned within a linear lumen 1 by an endoscope that checks a location of the procedure and a stent delivery system that delivers the stent to the location of the procedure.

Furthermore, when the covered stent 1000 expands the lesion portion 1a in the linear lumen 1, the expansion tube parts 150 are inserted into and caught by the lumen 1.

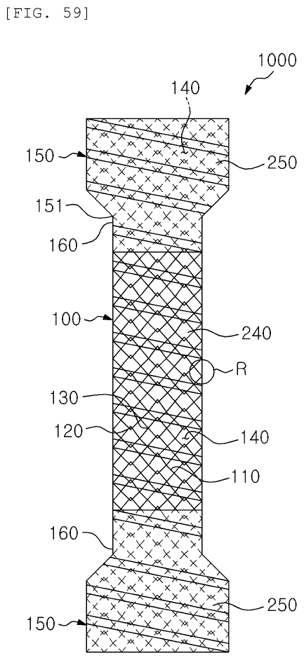

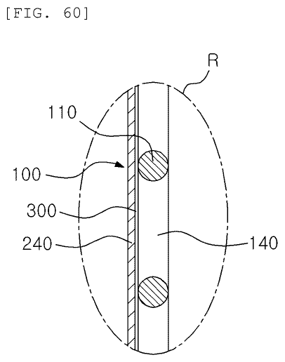

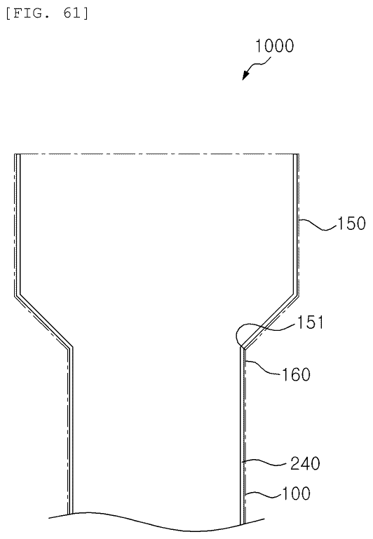

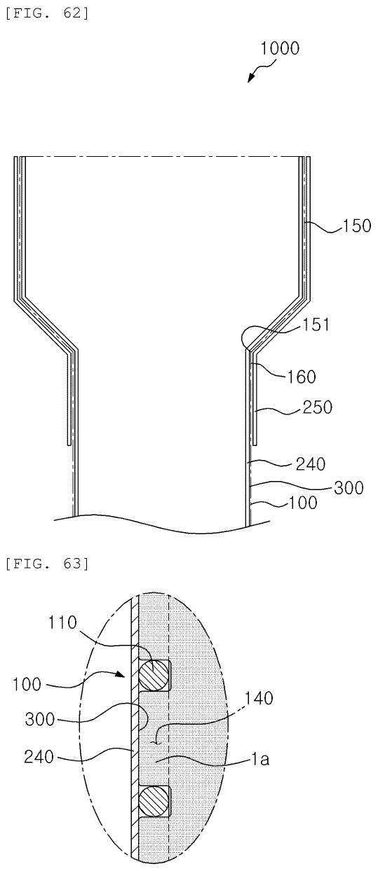

As shown in FIGS. 59 to 63, according to the first modification of the second embodiment of the present disclosure, the sixth film 240 of the covered stent 1000 may be formed to cover the inner surface of the body stent 100 and the inner surfaces of the expansion tube parts 150.

The seventh film 250 is formed to cover the outer surfaces of the expansion tube parts 150, the outer surfaces of the opposite end parts 160 of the body stent 100, and the opposite portions of the outer surface of the body stent 100 that are adjacent thereto.

Therefore, when the sixth and seventh films 240 and 250 are bonded, the gap space 300 is formed between a portion of the sixth film 240, which is not bonded to the seventh film 250, and the body stent 100.

At this point, the gap space 300 is formed between a portion of the sixth film 240, which is not bonded to the seventh film 250, and the inner surface of the body stent 100.

Furthermore, the space parts 140 located in the gap space 300 are exposed outwards.

Therefore, when the covered stent 1000 is positioned within the curved lumen 1, the lesion portion 1a intrudes into the space parts 140 located in the gap space 300.

For this reason, the space parts 140 of the covered stent 1000 are caught by the lesion portion 1a intruding into the stent.

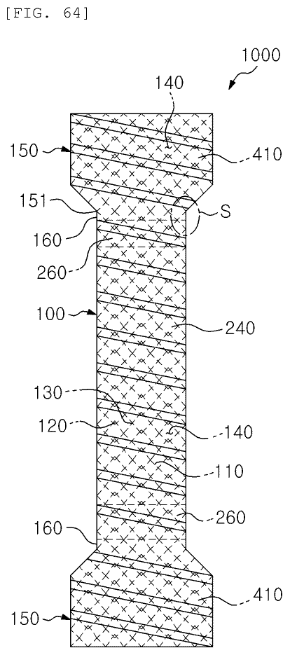

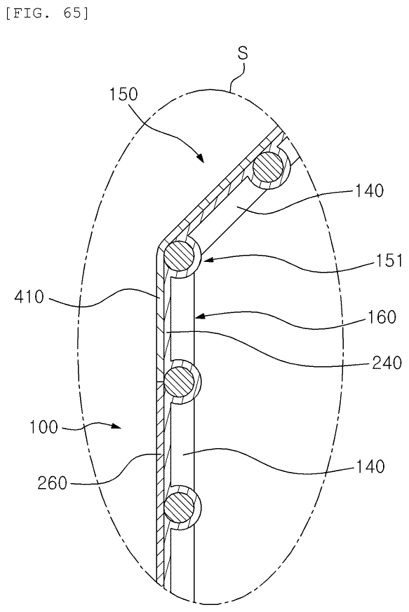

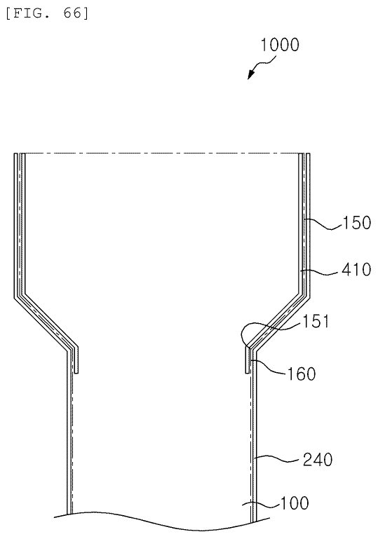

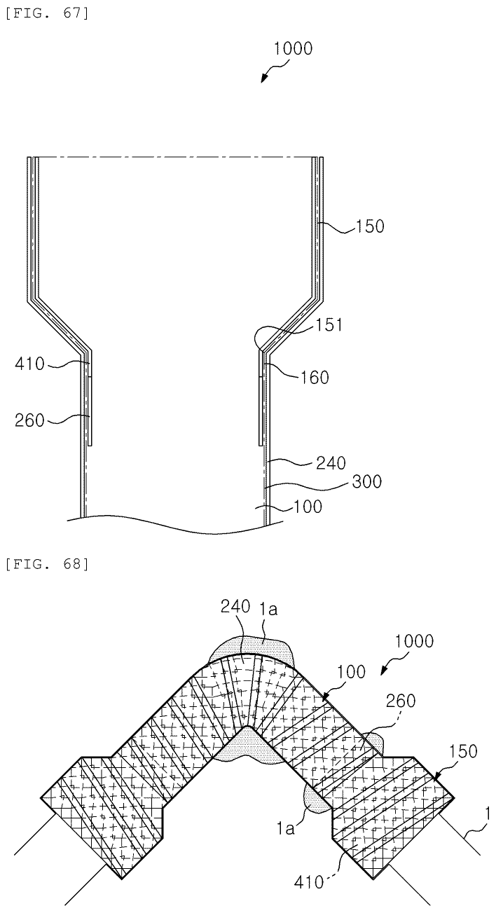

As shown in FIGS. 64 to 68, according to the second modification of the second embodiment of the present disclosure, the covered stent 1000 excludes the seventh film 250, and includes a silicone eighth film 410 and a ninth film 260 made of a PTFE material.

The silicone eighth film 410 is bonded to the inner surfaces of the expansion tube parts 150, the inner surfaces of the opposite end parts 160 of the body stent 100 adjacent to the bent parts 151 of the expansion tube parts 150 to be bonded to the sixth film 240.

The ninth film 260 made of a PTFE material covers the opposite portions of the inner surface of the body stent 100 adjacent to the inner surfaces of the opposite end parts 160 of the body stent 100 to be bonded to the sixth film 240.

At this point, the ninth film 260 may be made of a PTFE tape and have a spiral shape or a ring shape on the opposite portions of the inner surface of the body stent 100, which are adjacent to the inner surfaces of the opposite end parts 160 of the body stent 100, so that overlap portions are formed by overlapping each other.

Furthermore, the sixth, eighth, and ninth films 240, 410, and 260 prevent the space parts 140 from being exposed outwards.

Furthermore, the hook parts 120 and the cross parts 130 located at the bonded the sixth, eighth, and ninth films 240, 410, and 260 are fixed to the sixth, eighth, and ninth films 240, 410, and 260, so the hook parts 120 and the cross parts 130 are not free to be displaced.

The gap space 300 is formed between a portion of the sixth film 240, which is not bonded to the eighth and ninth films 410 and 260, and the body stent 100.

In other words, as the portion of the sixth film 240 not bonded to the eighth and ninth films 410 and 260 comes loose, the gap space 300 is formed between a portion of the sixth film 240 not bonded to the eighth and ninth films 410 and 260 and the outer surface of the body stent 100.

At this point, the hook parts 120 and the cross parts 130 located in the gap space 300 are not fixed to the fourth film 240 due to the gap space 300 and are free to be displaced.

Furthermore, a gap between the sixth, eighth, and ninth films 240, 410, and 260 is sealed by the bonded sixth, eighth, and ninth films 240, 410, and 260.

Therefore, when the covered stent 1000 is positioned within the curved lumen 1, the hook parts 120 and the cross parts 130 located in the gap space 300 are not fixed to the sixth film 240 by the gap space 300, so displacement occurs in the hook parts 120 and the cross parts 130 in correspondence to the curved lumen 1.

Furthermore, the space parts 140 are also deformed due to the displacement of the hook parts 120 and the cross parts 130.

For this reason, as shown in FIG. 68, the covered stent 1000 is easily deformed in a curved shape that fits the curved lumen 1 and expands the lesion portion 1a, thereby maintaining the passage of the lumen 1.

Furthermore, the covered stent 1000 is easily deformed in a curved shape to fit the curved lumen 1 without bending at the curved portion of the lumen 1.

Furthermore, the eighth film 410 made of a silicone material is formed on the bent parts 151 of the expansion tube parts 150 and the opposite end parts 160 of the body stent 100 to prevent the bent parts 151 of the expansion tube parts 150 from being bent by external force.

Furthermore, the expansion tube parts 150 of the covered stent 1000 are inserted in and caught by the lumen 1.

Furthermore, the expansion tube parts 150 are covered with the eighth film 410 made of a highly elastic silicone material and have strong resilience to maintain original forms thereof, so the expansion tube parts 150 are not easily compressed by external force such as movement of the human body.

Furthermore, the sixth and ninth films 240 and 260 made of a PTFE material and the eighth film 410 made of a silicone material may prevent the lesion portion 1a from intruding into the covered stent 1000.

Furthermore, a gap between the sixth, eighth, and ninth films 240, 410, and 260 is sealed when the sixth, eighth, and ninth films 240, 410, and 260 are bonded, thereby preventing the lesion portion 1a from intruding into the covered stent 1000.

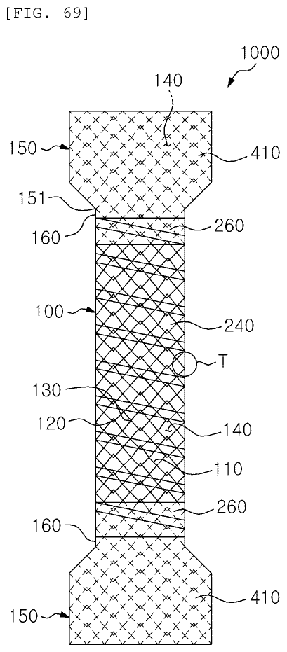

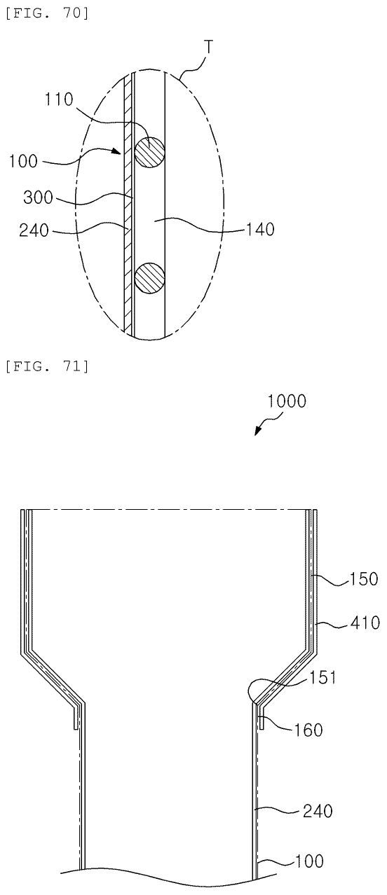

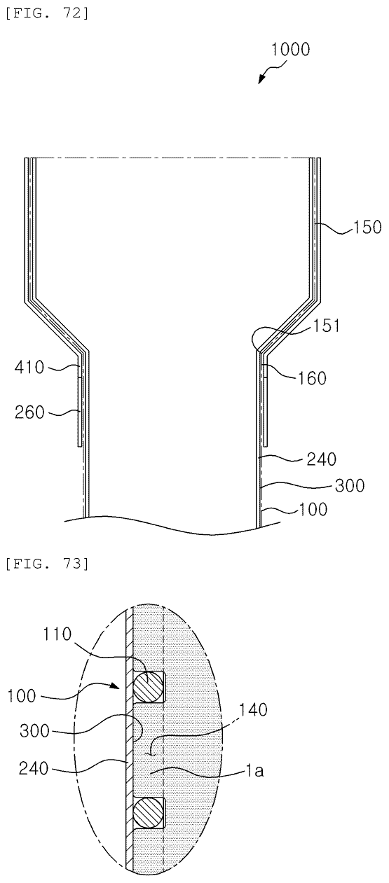

As shown in FIGS. 69 to 73, according to the third modification of the second embodiment of the present disclosure, the covered stent 1000 is configured to be the same as the second modification with different positions where the sixth, eighth, and ninth films 240, 410, and 260 of the second modification are installed.

The sixth film 240 is formed to cover the inner surface of the body stent 100 and the inner surfaces of the expansion tube parts 150.

The eighth film 410 is formed to cover the outer surfaces of the expansion tube parts 150 and the outer surfaces of the opposite end parts 160 of the body stent 100, which are adjacent to the bent parts 151 of the expansion tube parts 150, are covered with the eighth film 410 and bonded to the sixth film 240,

The ninth film 260 is formed to cover the opposite portions of the outer surface of the body stent 100 that are adjacent to the outer surfaces of the opposite end parts 160 of the body stent 100.

Therefore, when the sixth, eighth, and ninth films 240, 410, and 260 are thermally bonded, the gap space 300 is formed between a portion of the sixth film 240, which is not bonded to the eighth and ninth films 410 and 260, and the body stent 100.

At this point, the gap space 300 is formed between a portion of the sixth film 240, which is not bonded to the eighth and ninth films 410 and 260, and the inner surface of the body stent 100.

Furthermore, the space parts 140 located in the gap space 300 are exposed outwards.

Therefore, when the covered stent 1000 is positioned within the curved lumen 1, the lesion portion 1a intrudes into the space parts 140 located in the gap space 300.

For this reason, the space parts 140 of the covered stent 1000 are caught by the lesion portion 1a intruding into the stent.

Although the specific embodiments of the present disclosure have been provided, the present disclosure is not limited to the above-described embodiments, and various modifications, equivalents, additions and substitutions are possible by one of ordinary skill in the art to which this present disclosure belongs, without departing from the scope and spirit of the present disclosure.

What is claimed is:

1. A covered stent easily positioned within a curved lumen,
to expand a lesion portion (1a) that causes stenosis or obstruction in a lumen (1) in the body to maintain a passage of the lumen (1),
the covered stent comprising a cylindrical body stent (100) in which a plurality of hook parts (120) and a plurality of cross parts (130) are formed by weaving shape-memory alloy wires (110) to be caught in a hook shape or crossing the wires so that a plurality of space parts (140) is formed among the wires (110), and expansion tube parts (150) formed at opposite ends of the body stent (100) and having a larger diameter than a diameter of the body stent (100),
wherein a first film (200) made of a polytetrafluoroethylene (PTFE) material is formed to cover an outer surface of the body stent (100),
a second film (210) made of a polytetrafluoroethylene (PTFE) material is formed to cover outer surfaces of the expansion tube parts (150) and outer surfaces of opposite end parts (201) of the first film (200), which are adjacent to bent parts (151) of the expansion tube parts (150),
a third film (220) made of a polytetrafluoroethylene (PTFE) material is formed to cover inner surfaces of the expansion tube parts (150) and inner surfaces of opposite end parts (160) of the body stent (100), which are adjacent to the bent parts (151) of the expansion tube parts (150), and extend in a longitudinal direction of the body stent (100) longer than the second film (210), and
the first, second, and third films (200, 210, and 220) are thermally bonded, and a gap space (300) is formed between a portion of the first film (200), which is not bonded to the second and third films (210 and 220), and the body stent (100).

2. The covered stent of claim 1, wherein the first film (200) is formed to cover an inner surface of the body stent (100),
the third film (220) is formed to cover the inner surfaces of the expansion tube parts (150) and inner surfaces of the opposite end parts (201) of the first film (200),
the second film 210 is formed to cover the outer surfaces of the expansion tube parts (150) and outer surfaces of the opposite end parts (160) of the body stent (100) and extend in a longitudinal direction of the body stent (100) longer than the third film (220), and
the first, second, and third films (200, 210, and 220) are thermally bonded, and a gap space (300) is formed between a portion of the first film (200), which is not bonded to the second and third films (210 and 220), and the body stent (100).

3. The covered stent of claim 1, wherein, excluding the second and third films (210 and 220) from the covered stent,
the inner surfaces of the opposite end parts (160) of the body stent (100) and opposite portions of an inner surface of the body stent (100), which are adjacent to the opposite end parts, are covered with a fourth film (230) made of a polytetrafluoroethylene (PTFE) material and thermally bonded to the first film (200), and the gap space (300) is formed between a portion of the first film (200), which is not bonded to the fourth film (230), and the body stent (100), and
each of the expansion tube parts (150), an end part (231) of the fourth film (230), which is adjacent to each bent part (151) of the expansion tube parts (150), and the opposite end parts (201) of the first film (200) are covered with a fifth film (400) made of a silicone material by silicone dipping.

4. The covered stent of claim 3, wherein the first film (200) is formed to cover the inner surface of the body stent (100),
the fourth film (230) covers outer surfaces of the opposite end parts (160) of the body stent (100) and opposite portions of the outer surface of the body stent (100), which are adjacent to the opposite end parts, and the covered portions are thermally bonded to the first film (200), and the gap space (300) is formed between a portion of the first film 200, which is not bonded to the fourth film (230), and the body stent (100), and
the fifth film (400) covers each of the expansion tube parts (150), the end part (231) of the fourth film (230), and each of the opposite end parts (201) of the first film (200) by silicone dipping method.

5. The covered stent of claim 1, wherein the cross parts (130) are formed by between 20% and 80% of the covered stent, and
the hook parts (120) are formed by between 80% and 20% of the covered stent.

6. A covered stent easily positioned within a curved lumen,
to expand a lesion portion (1a) that causes stenosis or obstruction of a lumen (1) in the body to maintain a passage of the lumen (1),
the covered stent comprising a cylindrical body stent

(100) in which a plurality of hook parts (120) and a plurality of cross parts (130) are formed by weaving shape-memory alloy wires (110) to be caught in a hook shape or crossing the wires so that a plurality of space parts (140) is formed among the wires (110), and expansion tube parts (150) formed at opposite ends of the body stent (100) and having a larger diameter than a diameter of the body stent (100),
wherein a sixth film (240) made of a polytetrafluoroethylene (PTFE) material is formed to cover an outer surface of the body stent (100) and outer surfaces of the expansion tube parts (150), and
inner surfaces of the expansion tube parts (150), inner surfaces of the opposite end parts (160) of the body stent (100), which are adjacent to the bent parts (151) of the expansion tube parts (150), and opposite portions of an inner surface of the body stent (100), which is adjacent thereto, are covered with a seventh film (250) made of a polytetrafluoroethylene (PTFE) material and bonded to the sixth film (240), so a gap space (300) is formed between a portion of the sixth film (240), which is not bonded to the seventh film (250), and the body stent (100).

7. The covered stent of claim 6, wherein the sixth film (240) is formed to cover the inner surface of the body stent (100) and the inner surfaces of the expansion tube parts (150), and
the seventh film (250) covers the outer surfaces of the expansion tube parts (150), outer surfaces of the opposite end parts (160) of the body stent (100), and opposite portions of the outer surface of the body stent (100), which are adjacent to the opposite end parts, and the surfaces are bonded to the sixth film (240), so the gap space 300 is formed between a portion of the sixth film (240), which is not bonded to the seventh film (250), and the body stent (100).

8. The covered stent of claim 6, wherein excluding the seventh film (250) from the covered stent,
the inner surfaces of the expansion tube parts 150 and the inner surfaces of the opposite end parts (160) of the body stent (100), which are adjacent to the bent parts (151) of the expansion tube parts (150), are covered with a eighth film (410) made of a silicone material and bonded to the sixth film (240),
the opposite portions of the inner surface of the body stent

(100) which are adjacent to the inner surfaces of the opposite end parts (160) of the body stent (100), are covered with a ninth film (260) made of a polytetrafluoroethylene (PTFE) material and bonded to the sixth film (240), and
the gap space (300) is formed between a portion of the sixth film 240, which is not bonded to the eighth and ninth films (410 and 260), and the body stent (100).

9. The covered stent of claim 8, wherein the sixth film (240) is formed to cover the body stent (100) and the inner surfaces of the expansion tube parts (150),
the outer surfaces of the expansion tube parts (150) and the outer surfaces of the opposite end parts (160) of the body stent (100), which are adjacent to the bent parts (151) of the expansion tube parts (150), are covered with the eighth film (410) and bonded to the sixth film (240),
the opposite portions of the outer surface of the body stent (100) adjacent to the outer surfaces of the opposite end parts (160) of the body stent (100) are covered with the ninth film (260) and bonded to the sixth film (240), and
the gap space (300) is formed between a portion of the sixth film (240), which is not bonded to the eighth and ninth films (410 and 260), and the body stent (100).

10. The covered stent of claim 6, wherein the cross parts (130) are formed by between 20% and 80% of the covered stent, and
the hook parts (120) are formed by between 80% and 20% of the covered stent.
